(19) United States
(12) Patent Application Publication
(10) Pub. No.: US 2002/0168324 A1
(43) Pub. Date: Nov. 14, 2002

(54) Silica microbeads with sensory properties in the mouth, process for preparing them and toothpaste compositions containing them

(76) Inventors: Frederic Amiche, Aulnay-Sous-Bois (FR); Adrien Dromard, Lyon (FR); Yvonnick Chevallier, Fontaines St-Martin (FR); Pierre-Yves Lahary, Lyon (FR)

Correspondence Address: RHODIA INC., CN 7500, 259 Prospect Plains Road, CRANBURY, NJ, 08512, US

(21) Appl. No.: 10/100,398

(22) Filed: Mar. 18, 2002

Related U.S. Application Data
(63) Continuation of application No. 09/600,606, filed on Aug. 31, 2000, now abandoned.

(30) Foreign Application Priority Data
Jan. 20, 1998 (FR) 98/00564

(51) Int. Cl. A61K 7/16; A61K 7/18

(52) U.S. Cl. 424/49; 424/52

(57) ABSTRACT

The invention relates to precipitated silica microbeads having sensory properties in the mouth, and a specific surface CTAB of at least 100 m²/g, an oil uptake of at least 200 ml/g, a mean particle diameter from 50 μm to 600 μm, at the surface from 0.5 to 2 parts by weight, (expressed as zinc metal) of a zinc derivative with a degree of oxidation of 2 per 100 parts by weight of silica, a pH from about 7 to 9, and possibly between 0.2 and 5 parts by weight of a white pigment for 100 parts by weight of silica The invention also relates to a method for producing said microbeads by precipitation, treatment of the silica slurry with a soluble zinc compound, mechanical thinning of the cake resulting from the separation of the treated silica slurry, and drying by atomization. The invention further relates to their use in toothpaste preparations as sensory agents acting in the mouth.

The present invention relates to silica microbeads with sensory properties in the mouth, to a process for preparing them, to their use as sensory additives in toothpaste compositions and to the toothpaste compositions containing said microbeads.

The mineral fillers used in toothpastes generally have particle sizes of less than 50 μm and do not provide any specific sensory effect.

The idea of introducing coarse mineral particles (at least 100 μm) of limited cohesion into toothpastes, providing a sensory effect by disintegration on brushing, is known.

U.S. Pat. No. 4,871,396 describes the introduction into toothpastes of granules which disintegrate on brushing, obtained by atomizing an aqueous dispersion of at least two inorganic powders (zeolite and colloidal silica in particular), the colloidal silica having the function of a binder.

Patent application GB-A-2 272 640 describes granules from 0.03 mm to 3 mm based on thickening fumed silica which are preferably obtained by granulation, and their use in toothpastes.

Patent applications WO 96/09033 and WO 96/09034 describe granules from 40 μm to 600 μm obtained by granulating a mixture of two types of silica, so-called abrasive silica, on the one hand, and so-called thickening silica, on the other hand, and the use of these granules in toothpastes; the cohesion of the granules is adjusted by the abrasive silica/thickening silica ratio.

Fumed silica microbeads defined by characteristics of BET and CTAB specific surface of at least 100 m²/g and a DOP oil uptake of at least 200 ml/100 g, obtained by atomization, are known as reinforcing fillers in elastomers.

The production of these microbeads requires the use, in order to carry out the atomization operation, of a silica suspension with a high solids content, generally of at least 15%. Such solids contents are generally obtained by compacting the filter cakes and resuspending (known as fluidization or crumbling) in order to obtain a level of viscosity which is compatible with an atomization operation.

It has been proposed to carry out this fluidization or crumbling operation by wet grinding (FR-A-2 453 880), by acidification (FR-A-2 453 880) or by addition of aluminum salts (FR-A-2 536 380).

Crumbling by wet grinding has the drawback of an insufficient production efficiency and a lack of stability of the crumbled suspensions leaving the grinder. Crumbling by acidification leads to microbeads whose cohesion is difficult to control; crumbling by addition of aluminum salts leads to microbeads which react with the fluoride ions and other anti-caries agents present in the toothpastes.

In addition, these microbeads have the drawback of having a low refractive index and thus of not being sufficiently visible in the toothpaste compositions.

It is known practice to treat or functionalize the surface of silica particles with water-soluble zinc derivatives in order to modify the interactions or the reactivity of this silica with the medium in which it is formulated.

Thus, patent application FR-A-2 632 185 describes the modification of abrasive or thickening fumed silica of small particle size (from 1 μm to 10 μm) with an alkaline zincate in order to improve the compatibility of the silica with toothpaste formulations containing zinc derivatives.

Patent EP-A-356 406 describes the modification, with zinc chloride, of the surface of fillers for paper which consist of particles formed of a calcium carbonate core and silica shell, in order to make them insensitive to acidic media.

Patent applications WO 96/30302 and WO 96/30301 are directed toward silica microbeads of coarse particle size, used for reinforcing elastomers, said microbeads having been modified by a treatment with a zinc compound (zinc sulfate) in large amount (1 to 5 parts by weight expressed as Zn metal per 100 parts by weight of silica SiO₂), followed by a crumbling step preferably carried out in the presence of an aluminum salt. In a toothpaste formulation, such microbeads are said to have the drawbacks of an insufficient visibility on account of their low refractive index, and poor compatibility with fluoride ions on account of the preferential presence of aluminum salt.

The Applicant has found silica microbeads which are nonabrasive or only weakly abrasive, which, on account of their better shape factor (spherical particles, with few or no corners, of uniform size distribution) and their controlled cohesion provided by the presence of a small amount of a zinc derivative at the surface, provides toothpastes with a specific sensory effect which is better than that imparted by thickening silica granules of the same diameter or by granules of a mixture of abrasive and thickening silica. In addition, these microbeads are compatible with the other ingredients of the toothpaste formulation.

Moreover, the precipitation preparation process used allows the addition during the synthesis of pigments, in particular white pigments, such as titanium dioxide or zinc oxide, which improves the visibility of the silica microbeads in the toothpaste and do so without degrading the cohesion of said microbeads.

A first subject of the invention consists of fumed silica microbeads which can be used as sensory additives in toothpastes, characterized in that they have:

a CTAB specific surface of at least 100 m²/g, preferably from about 120 m²/g to 250 m²/g and most particularly from about 140 m²/g to 200 m²/g

a DOP oil uptake of at least 200 ml/g, preferably from about 200 ml/g to 350 ml/g

a median particle diameter from 50 μm to 600 μm, generally from about 100 μm to 400 μm

at the surface from 0.5 to 2 parts by weight, preferably from 0.5 to less than 2 parts by weight, most particularly from 0.6 to 1.5 parts by weight (expressed as zinc metal) of a zinc derivative of oxidation state “2” per 100 parts by weight of silica (SiO₂)

a pH from about 7 to 9, preferably from about 7.5 to 8.7.

The CTAB specific surface is the external surface determined according to NFT standard 45007 (November 1987).

The DOP oil uptake is determined according to ISO standard 787/5 using dioctyl phthalate.

The mean weight diameter d₅₀ of the silica particles is determined using a Sympatec Helos machine. This machine applies the principle of Fraunhoffer scattering and uses a low power He/Ne laser. The sample is predispersed by mechanical stirring, without ultrasound, in water for 30 seconds in order to obtain an aqueous suspension.

The granulometric analysis is also carried out by dry screening according to standard NF X 11-507.

The pH is measured according to ISO standard 787/9 (pH of a 5% by weight suspension in deionized water).

Said microbeads also have a BET specific surface of at least 100 m²/g, preferably from about 120 m²/g to 300 m²/g, most particularly from about 140 m²/g to 250 m²/g.

The BET specific surface is determined according to the Brunauer-Emmet-Teller method described in the “Journal of the American Chemical Society”, vol. 60, page 309, February 1938 and corresponding to ISO standard 5794/1 (Annex D).

The silica microbeads of the invention have the property of disintegrating in an adequate manner in a toothpaste composition during a brushing operation, thus providing a sensory effect in the mouth.

One method for evaluating this effect can be to quantify the cohesion of the silica microbeads using a specific test of cohesion by mechanical stirring.

This test makes it possible to evaluate the change in the median diameter d₅₀ of a silica suspension, by particle size measurement before and after 10 minutes of mechanical stirring (without using ultrasound).

According to this test, the particle size measurement (by laser scattering on a Laser Sympatec Helos granulometer), is carried out on a silica suspension having an optical concentration of 20%±3%, which is mechanically stirred (without using ultrasound).

After 30 s of mechanical dispersion of the suspension in the granulometer tank, the initial median diameter d₅₀ᵢ is measured.

After stirring for 10 minutes, the level of the tank having been readjusted to maintain an optical concentration of 20%±3% in the dispersion, the final median diameter d_{50f} is measured.

The more the diameters after mechanical stirring remain close to the initial diameters, the more cohesive the particles.

The results will be all the more comparative the closer the sizes of the initial particles. For this, screening of the particles will be carried out, if necessary.

The cohesion factor CF, expressed as a percentage, based on the initial d₅₀ (d₅₀ᵢ) and final d₅₀ (d_{50f}) median diameters is calculated according to the following equation:

CF=(d_{50f}/d₅₀ᵢ)×100

According to this test, which is described later in greater detail, carried out on a fresh product, the microbeads of the invention have, for an initial median particle diameter d₅₀ᵢ of about 180 μm±10 μm, a cohesion factor from 50% to less than 90%, more generally from 55% to 80%.

These microbeads also have the property of being compatible with the fluorinated additives (NaF, monofluorophosphates, amine fluorides, etc.) generally present in toothpastes; their compatibility is greater than 75%, preferably greater than 85%, with NaF.

The compatibility of the microbeads (referred to as “silica” in the tests) with sodium fluoride (NaF) can be measured according to a test, the principle of which consists:

in leaving the test silica in contact with an aqueous solution of sodium fluoride and of phosphates of known concentration, for 1 hour at 60° C.

in measuring, by ionometry, the concentration of fluoride ion [unreacted F⁻] present in the liquid medium obtained by centrifugation

and in comparing this concentration with the fluoride ion concentration [starting F⁻] of the initial solution which has not had any contact with the silica.

The compatibility with NaF, expressed as a percentage, is obtained by the following calculation:

([unreacted F⁻]/[starting F⁻])×100

This test is described later in greater detail.

Said silica microbeads preferably also comprise at least one mineral pigment, in particular a white mineral pigment, such as titanium dioxide, zinc oxide, etc., in an amount from about 0.2 to 5 parts by weight, preferably from 0.5 to 4 parts by weight (expressed as weight of pigment, in particular metal oxide) per 100 parts by weight of silica (SiO₂).

Said microbeads can be obtained according to a precipitation process, comprising the steps for formation of an aqueous silica slurry by reacting a silicate of an alkali metal M, with an SiO₂/M₂O ratio from about 2 to 4, with an acidifying agent, separation of the silica slurry formed, washing, fluidization (crumbling) of the silica cake recovered and drying, said process being characterized in that:

the crumbling operation is carried out on a silica cake with a solids content of at least about 15% by weight, said cake resulting from the separation of a silica slurry treated with from 0.5 to 2 parts by weight, preferably from 0.5 to less than 2 parts by weight, most particularly from 0.6 to 1 part by weight (expressed as zinc metal) of at least one acidic or basic soluble zinc compound with an oxidation state of 2 per 100 parts by weight of silica (SiO₂), and performed using a basic agent (in the case of a treatment using at least acidic zinc compound) or acidic agent (in the case of a treatment using at least one basic zinc compound), respectively, at a pH value from about 7.5 to 9.5, preferably from about 7.5 to 9

and the drying operation is carried out by atomization.

The choice of the silicate and of the acidifying agent to carry out the step for formation of the silica slurry according to the process of the invention is made in a manner which is well known per se.

The alkali metal silicate is advantageously a sodium or potassium silicate. Sodium silicates may be mentioned most particularly.

Said silicate is used in the form of an aqueous solution having a concentration expressed as SiO₂ from about 20 g/l to 350 g/l, preferably from about 100 g/l to 260 g/l.

A strong inorganic acid such as sulfuric acid, nitric acid or hydrochloric acid or an organic acid such as acetic acid, formic acid or carbonic acid is generally used as acidifying agent. It is preferably sulfuric acid. This acid can be used in dilute or concentrated form, preferably in the form of an aqueous solution having a concentration from about 60 g/l to 400 g/l.

The step for formation of the silica slurry can be carried out according to various known methods at a temperature of at least 60° C., preferably from about 70° C. to 98° C., most particularly from 80° C. to 98° C.

Thus, this step can be carried out by gradual neutralization of a tail stock consisting of an aqueous alkali metal silicate solution optionally containing an electrolyte, by continuous or batchwise addition of an acid. Such a procedure is described in particular in the Examples 1 of documents FR-A-1 054 175 and U.S. Pat. No. 2,940,830.

Another method can consist in simultaneously introducing a solution of alkali metal silicate and of acid onto a tail stock consisting of:

water optionally with an acid or base added, with a pH from about 4 to 11

or an aqueous alkali metal silicate solution optionally containing an electrolyte, optionally partially neutralized with an acid, or a silica suspension (slurry), with a pH from about 6 to 9 while maintaining a substantially constant pH from about 7 to 9 in the medium during the simultaneous introduction of the reagents, and then in introducing, if desired, an acid until a pH from about 3 to 6 is obtained. This final operation of introduction of acid is aimed at facilitating the subsequent filtration and washing operations; this operation can be partially or totally omitted when the treatment with the soluble zinc compound is carried out using an acidic zinc salt.

Among the electrolytes which may be present in the tail stock, mention may be made in particular of the soluble salts of alkali metals or alkaline-earth metals, in particular the metal salt of the starting silicate and of the acidifying agent, i.e. preferably sodium sulfate in the case of the reaction of a sodium silicate with sulfuric acid. The amount of electrolyte present can be from about 0.1 mol to 1 mol of electrolytic alkali metal salt or from about 10 mmol to 100 mmol of electrolytic alkaline-earth metal salt per liter of tail stock. In the case of sodium sulfate, this amount can be up to 17 g/l, preferably up to 14 g/l of tail stock.

Procedures for neutralization by simultaneous introduction of silicate and acid into a tail stock are described in particular in EP-A-18 866; EP-A-396 450; EP-A-520 862; FR-A-2 710 629 and EP-A-670 813.

Among the soluble zinc compounds of oxidation state “2” used to carry out the treatment operation, mention may be made of:

acidic compounds such as inorganic or organic salts such as the halides (chloride), oxyhalides (oxychloride), nitrate, sulfate or carboxylates (acetate)

basic compounds such as zincates containing ZnO₂²⁻, HZnO₂⁻, Zn₂O₄⁴⁻ or ZnO₄⁶⁻ anions, more particularly alkali metal zincates, in particular sodium zincate; (the method for preparing the alkali metal zincates by reacting alkaline solutions with a zinc oxide is described in FR-A-2 632 185).

In practice, the acidic or basic zinc compound can be used in the form of a solution, generally an aqueous solution.

The treatment with an acidic zinc compound of oxidation state “2” is followed or accompanied by the addition of a basic agent such as aqueous ammonia, sodium hydroxide, alkaline silicates, etc., preferably in a aqueous solution.

The treatment with a basic zinc compound of oxidation state “2” is followed by the addition of an acidic agent such as nitric acid, sulfuric acid, hydrochloric acid, carbonic acid, etc., preferably in aqueous solution.

Said treatment with at least one soluble zinc compound and the basic or acidic agent for precipitating the zinc derivative can be carried out either on the slurry during precipitation, or at the end of precipitation and/or after precipitation, before the filtration/washing step.

A first embodiment of the treatment operation with the soluble zinc compound consists in adding a to silica slurry with a pH from about 7 to 9, preferably from about 7.5 to 8.5, at the end and/or after precipitation at a temperature from about 15° C. to 95° C., an aqueous solution of a soluble acidic zinc salt of oxidation state “2” (for example zinc sulfate), followed by a basic agent (for example sodium hydroxide), until a pH from about 7.5 to 9.5, preferably from about 7.5 to 9, is obtained. After optional maturation (for example from 5 minutes to 2 hours), the treated silica slurry is then filtered and washed until a silica cake with a solids content generally of at least 15% by weight is obtained. Silica microbeads are then obtained by mechanical crumbling and spray-drying.

A second embodiment of the treatment operation with the soluble zinc compound consists in simultaneously adding to a silica slurry undergoing precipitation, with a pH from about 7 to 9, preferably from about 7.5 to 8.5, a silicate solution and a solution of acidic zinc salt (for example sulfate). After optional maturation (for example from 10 minutes to 1 hour), the treated silica slurry is then filtered and washed until a silica cake with a solids content generally of at least 15% by weight is obtained. Silica microbeads are then obtained by mechanical crumbling and spray-drying.

Said treatment is preferably carried out on a silica slurry according to the first embodiment described above, using a soluble acidic zinc compound, in particular using zinc sulfate in aqueous solution.

The step of separation of the treated slurry is carried out by filtration or by any other means; this operation is preferably coupled with a washing operation.

The steps of filtration/washing of the treated slurry are preferably carried out using a filter press and water as washing agent, in order to obtain a silica cake of the desired solids content, of at least 15% by weight, preferably of at least 16% by weight.

The step of crumbling (i.e. of fluidization) of the cake can be carried out by the simple action of mechanical stirring using a shearing rotor, optionally with addition of water.

The Applicant has observed, and this constitutes one of the advantages of the invention, that the presence of zinc at the surface of the silica makes the crumbling operation of the silica cake particularly easy, without the need to use chemical means (for example addition of aluminum salt) that are incompatible with applications as toothpastes.

The atomization step is preferably carried out using a nozzle sprayer.

The microbeads thus obtained have a spherical appearance and have a median diameter from about 50 μm to 600 μm, generally from about 100 μm to 400 μm.

One method for preparing the microbeads that are the subject of the invention which most particularly performs well consists in carrying out, under the conditions mentioned above,

an operation for formation of a silica slurry by reaction of an alkali metal silicate and an acidifying agent, according to the following steps:

a first step consisting in using an initial tail stock consisting of water, alkali metal silicate and an electrolytic salt, the concentration of alkali metal silicate (expressed as SiO₂) in said tail stock possibly being up to 100 g/l

a second step consisting in neutralizing said tail stock with the acidifying agent until a pH of greater than or equal to about 7, preferably from about 7 to 9.5 and most particularly from about 7 to 8.5, is obtained in the reaction medium;

a third step consisting in introducing into said neutralized tail stock the alkali metal silicate in aqueous solution and the acidifying agent, under conditions such that the pH of the reaction medium remains substantially constant and above or equal to about 7, preferably from about 7 to 9.5 and most particularly from about 7 to 8.5;

an operation for treatment of the slurry obtained, after optional maturation, by addition of 0.5 to 2 parts, preferably from 0.5 to less than 2 parts and most particularly from 0.6 to 1.5 parts (expressed as zinc metal), of at least one soluble acidic zinc compound of oxidation state “2” per 100 parts of silica, and then, after optional maturation, addition of a basic agent, until a pH from about 7.5 to 9.5, preferably from about 7.5 to 9, is obtained in the reaction medium, and optional maturation;

optionally, an operation to adjust the pH to a value from about 7 to 8.5 by addition of an acidic agent, and optional maturation;

an operation of separation and washing so as to obtain a silica cake with a solids content of at least 15% by weight, preferably of at least 16% by weight, in particular using a filter press;

an operation for crumbling by means of mechanical stirrers;

and a spray-drying operation.

One variant of the preparation process for preparing the silica microbeads of the invention consists in introducing at any point in the synthesis of the silica slurry (either into the tail stock or during or after precipitation of the silica) or, preferably, during the step of crumbling of the silica cake containing zinc, from about 0.2 to 5 parts by weight, preferably from about 0.5 to 4 parts by weight per 100 parts by weight of silica, expressed as SiO₂, of at least one mineral pigment, in particular a white pigment such as titanium dioxide or zinc oxide.

The subject of the present invention is also the use of the silica microbeads forming the subject of the invention or obtained according to the process of the invention, as sensory agents in the mouth, in toothpaste compositions, as well as the toothpaste compositions comprising said silica.

Said silica can be present in said toothpaste compositions in a proportion from about 0.5% to 20%, preferably from about 1% to 15%, of the weight of said compositions.

These compositions can also comprise other usual ingredients, in particular water-insoluble mineral abrasive agents, thickeners, wetting agents, etc.

Abrasive agents which may be mentioned in particular include abrasive silicas, calcium carbonate, hydrated alumina, bentonite, aluminum silicate, zirconium silicate, and sodium, potassium, calcium and magnesium metaphosphates and phosphates. The total amount of abrasive powder(s) can constitute from about 5% to 50% of the weight of the dental composition.

Among the thickeners which may be mentioned most particularly are thickening silicas in an amount from about 1% to 15% of the weight, xanthan gum, guar gum, carrageenans, cellulose derivatives or alginates, in an amount which can be up to 5% of the weight of said composition.

Among the wetting agents which may be mentioned, for example, are glycerol, sorbitol, polyethylene glycols, polypropylene glycols and xylitol, in an amount from about 2% to 85%, preferably from about 3% to 55%, of the weight of the toothpaste composition expressed as solids.

These toothpaste compositions can also comprise surfactants, detergents, dyes, antibacterial agents, fluorinated derivatives, opacifiers, flavorings, sweeteners, antitartar agents, antiplaque agents, bleaching agents, sodium bicarbonate, antiseptics, enzymes, natural extracts (camomile, thyme, etc.) etc.

The examples which follow are given for illustrative purposes.

Measurements of the Average Size and Cohesion of the Microbeads

The apparatus used is a Sympatec laser scattering granulometer, equipped with Helos software, a Sucell cl 245 liquid dispersion system and a 500 mm analytical focal length. For all measurements, the optical concentration of the silica suspension in the granulometer tank is adjusted to 20±3%.

The initial particle size of the microbeads, expressed as d₁₀, d₅₀ and d₉₀ diameters, is measured after 30 seconds under mechanical stirring at a speed corresponding to position 8 of the machine.

The measurement of the cohesion of the microspheres is made with the same apparatus, under the same conditions, after 5 minutes, 10 minutes, 15 minutes and 20 minutes of stirring (the optical concentration of the silica suspension in the granulometer tank being adjusted, if necessary, to 20±3% by dilution). The results are also expressed as d₁₀, d₅₀ and d₉₀ diameters.

The more the diameters after mechanical stirring remain close to the initial diameters, the more cohesive the particles.

Since the results are all the more comparative the closer the sizes of the initial particles, a screening of the particles is carried out for this purpose, if necessary.

The cohesion factor CF, expressed as a percentage, is based on the initial median d₅₀ diameters (d₅₀ᵢ) and the median d₅₀ diameters after 10 minutes (d_{50f}) and calculated according to the following equation:

CF=(d_{50f}/d₅₀ᵢ)×100

Measurement of the Compatibility with NaF:

Preparation of the “Measurement Solution”

A stock solution containing 11.2 g of NaF, 86 g of NaH₂PO₄.H₂O, 333.6 g of Na₂HPO₄.2H₂O and 2690 g of deionized water is first prepared.

7 g of silica and 30 g of the stock solution prepared are then stirred at 60° C. for 1 hour in a hermetically sealed flask.

After cooling to room temperature, the supernatant is recovered after centrifugation.

The “measurement solution” finally comprises 10 ml of this supernatant and 25 ml of TAFIC buffer from Radiometer Analytical S.A. (of pH=5.3, consisting of 1M NaCl, 0.25M CH₃COOH, 0.75M CH₃COONa and 0.01M cyclohexanediaminetetraacetic acid).

Preparation of the “Control Solution”

The “control solution” comprises 10 ml of the stock solution prepared above and 25 ml of TAFIC buffer.

Assay

The assay is carried out using an electrode which is specific for fluorine ions (Tacussel PF4L1), a Calomel reference electrode (TR100) and an ionometer (Tacussel PHM 95).

The compatibility with NaF, expressed as a percentage, is obtained by the following calculation:

([unreacted F⁻]/[starting F⁻])×100

[unreacted F⁻] representing the concentration of unreacted fluoride ion in the “measurement solution” [starting F⁻] representing the concentration of fluoride ion in the “control solution”.

### EXAMPLE 1

The following are introduced into a stainless steel reactor fitted with an impeller stirring system and a heating jacket:

660 liters of water

11.8 kg of Na₂SO₄ (electrolyte)

323 liters of aqueous sodium silicate, with an SiO₂/Na₂O weight ratio equal to 3.45 and a density at 20° C. equal to 1.230.

The SiO₂ concentration in the tail stock is 77 g/l. The mixture is brought to a temperature of 82° C. while stirring is continued. 395 liters of dilute sulfuric acid with a density at 20° C. equal to 1.050 are introduced therein until a pH value (measured at its temperature) equal to 7.5 is obtained in the reaction medium. The reaction temperature is 82° C. during the first 15 minutes of the reaction; it is then raised from 82° C. to 95° C. over about 15 minutes, and then maintained at 95° C. until the end of the reaction.

77 liters of aqueous sodium silicate of the type described above and 106 liters of sulfuric acid, also of the type described above, are then introduced simultaneously into the reaction medium, this simultaneous introduction of acid and silicate being performed such that the pH of the reaction medium during the introduction period is constantly equal to 7.5±0.1.

An aqueous solution containing 85 g/l of ZnSO₄.7H₂O is then introduced at a flow rate of 140 l/h over 12 minutes. An aqueous solution containing 180 g/l of NaOH is then introduced so as to bring the pH value to 8. When this value is reached, introduction of the NaOH solution is stopped and the reaction slurry is left stirring for 10 min. Dilute sulfuric acid with a density at 20° C. equal to 1050 kg/m³ is then introduced, so as to bring the pH value to 7. The reaction slurry is then maintained at this pH for 5 minutes. The total reaction time is 126 minutes.

A reaction suspension is thus obtained which is filtered and washed by means of a filter press, such that a silica cake with a solids content of 19.4% is recovered.

This cake is then fluidized by mechanical action in a crumbler equipped with a central stirrer comprising four twin-paddles and a peripheral stirrer of scraper type. During this fluidization operation 1.7 kg of powdered titanium oxide are introduced into the crumbler. After this crumbling operation, a pumpable cake with a pH equal to 8.1 and a loss on ignition equal to 80% is obtained; this cake is dried using a nozzle sprayer. The characteristics of the silica microbeads obtained are as follows:

| | | |
|---|---|---|
| content of Zn metal | 0.5% | |
| BET specific surface | 180 | m²/g |
| CTAB specific surface | 164 | m²/g |
| DOP oil uptake | 260 | ml/100 g |
| pH | 8.2 | |
| Na₂SO₄ | 0.7% | |
| content of TiO₂ | 1.7% | |

The particle size characteristics obtained by dry-screening are as follows:

| Mesh size in μm | 250 | 210 | 180 | 150 | 125 |
|---|---|---|---|---|---|
| Cumulative oversize in | 3 | 15 | 35 | 56 | 76 |
| % weight/weight | | | | | |

The particle size characteristics (Laser determination) of the particles obtained are as follows:

| | | |
|---|---|---|
| d₁₀ = | 72 | μm |
| d₅₀ = | 158 | μm |
| d₉₀ = | 256 | μm. |

After dry-screening, the results of the cohesion test are as follows:

| Mechanical | d₁₀ | d₅₀ | d₉₀ |
|---|---|---|---|
| stirring time | (μm) | (μm) | (μm) |
| 30 seconds | 85 | 178 | 277 |
| 5 minutes | 16 | 119 | 224 |
| 10 minutes | 13 | 100 | 203 |
| 15 minutes | 11 | 83 | 176 |
| 20 minutes | 10 | 77 | 168 |

CF = 56.2%
compatibility with NaF: 96%

### EXAMPLE 2

The following are introduced into a stainless steel reactor fitted with an impeller stirring system and a heating jacket:

660 liters of water

11.8 kg of Na₂SO₄ (electrolyte)

323 liters of aqueous sodium silicate, with an SiO₂/Na₂O weight ratio equal to 3.45 and a density at 20° C. equal to 1.230.

The SiO₂ concentration in the tail stock is 77 g/l. The mixture is brought to a temperature of 82° C. while stirring is continued. 395 liters of dilute sulfuric acid with a density at 20° C. equal to 1.050 are introduced therein until a pH value (measured at its temperature) equal to 7.5 is obtained in the reaction medium. The reaction temperature is 82° C. during the first 15 minutes of the reaction; it is then raised from 82° C. to 95° C. over about 15 minutes, and then maintained at 95° C. until the end of the reaction.

77 liters of aqueous sodium silicate of the type described above and 106 liters of sulfuric acid, also of the type described above, are then introduced simultaneously into the reaction medium, this simultaneous introduction of acid and silicate being performed such that the pH of the reaction medium during the introduction period is constantly equal to 7.5±0.1.

An aqueous solution containing 170 g/l of ZnSO₄.7H₂O is then introduced at a flow rate of 140 l/h over 12 minutes. An aqueous solution containing 180 g/l of NaOH is then introduced so as to bring the pH value to 8. When this value is reached, introduction of the NaOH solution is stopped and the reaction slurry is left stirring for 10 min. Dilute sulfuric acid with a density at 20° C. equal to 1050 kg/m³ is then introduced, so as to bring the pH value to 7. The reaction slurry is then maintained at this pH for 5 minutes. The total reaction time is 128 minutes.

A reaction suspension is thus obtained which is filtered and washed by means of a filter press, such that a silica cake with a solids content of 20.1% is recovered.

This cake is then fluidized by mechanical action in a crumbler equipped with a central stirrer comprising four twin-paddles and a peripheral stirrer of scraper type. During this fluidization operation 1.7 kg of powdered titanium oxide are introduced into the crumbler. After this crumbling operation, a pumpable cake with a pH equal to 8.4 and a loss on ignition equal to 79.5% is obtained; this cake is dried using a nozzle sprayer. The characteristics of the silica microbeads obtained are as follows:

| | | |
|---|---|---|
| content of Zn metal | 1% | |
| BET specific surface | 174 | m²/g |
| CTAB specific surface | 164 | m²/g |
| DOP oil uptake | 260 | ml/100 g |
| pH | 8.7 | |
| Na₂SO₄ | 0.7% | |
| content of TiO₂ | 1.7% | |

The particle size characteristics obtained by dry-screening are as follows:

| Mesh size in μm | 250 | 210 | 180 | 150 | 125 |
|---|---|---|---|---|---|
| Cumulative oversize in | 7 | 21 | 43 | 65 | 81 |
| % weight/weight | | | | | |

The particle size characteristics (Laser determination) of the particles obtained are as follows:

| | | |
|---|---|---|
| d₁₀ = | 96 | μm |
| d₅₀ = | 174 | μm |
| d₉₀ = | 279 | μm. |

The results of the cohesion test are as follows:

| Mechanical | d₁₀ | d₅₀ | d₉₀ |
|---|---|---|---|
| stirring time | (μm) | (μm) | (μm) |
| 30 seconds | 96 | 174 | 279 |
| 5 minutes | 61 | 147 | 244 |
| 10 minutes | 49 | 135 | 228 |
| 15 minutes | 42 | 125 | 213 |
| 20 minutes | 35 | 118 | 205 |

CF = 77.6%
compatibility with NaF: 90%

### EXAMPLE 3

The following are introduced into a stainless steel reactor fitted with an impeller stirring system and a heating jacket:

660 liters of water

11.8 kg of Na₂SO₄ (electrolyte)

323 liters of aqueous sodium silicate, with an SiO₂/Na₂O weight ratio equal to 3.45 and a density at 20° C. equal to 1.230.

The SiO₂ concentration in the tail stock is 77 g/l. The mixture is brought to a temperature of 82° C. while stirring is continued. 395 liters of dilute sulfuric acid with a density at 20° C. equal to 1.050 are introduced therein until a pH value (measured at its temperature) equal to 7.5 is obtained in the reaction medium. The reaction temperature is 82° C. during the first 15 minutes of the reaction; it is then raised from 82° C. to 95° C. over about 15 minutes, and then maintained at 95° C. until the end of the reaction.

77 liters of aqueous sodium silicate of the type described above and 106 liters of sulfuric acid, also of the type described above, are then introduced simultaneously into the reaction medium, this simultaneous introduction of acid and silicate being performed such that the pH of the reaction medium during the introduction period is constantly equal to 7.5±0.1.

An aqueous solution containing 136 g/l of ZnSO₄.7H₂O is then introduced at a flow rate of 140 l/h over 12 minutes. An aqueous solution containing 180 g/l of NaOH is then introduced so as to bring the pH value to 8. When this value is reached, introduction of the NaOH solution is stopped and the reaction slurry is left stirring for 10 min. Dilute sulfuric acid with a density at 20° C. equal to 1050 kg/m³ is then introduced, so as to bring the pH value to 7. The reaction slurry is then maintained at this pH for 5 minutes. The total reaction time is 128 minutes.

A reaction suspension is thus obtained which is filtered and washed by means of a filter press, such that a silica cake with a solids content of 21.7% is recovered.

This cake is then fluidized by mechanical action in a crumbler equipped with a central stirrer comprising four twin-paddles and a peripheral stirrer of scraper type. During this fluidization operation 1.7 kg of powdered titanium oxide are introduced into the crumbler.

After this crumbling operation, a pumpable cake with a pH equal to 8.2 and a loss on ignition equal to 78.8% is obtained.

This cake is divided into 2 parts.

The first part is dried using a nozzle sprayer.

The characteristics of the silica microbeads obtained are as follows:

| | | |
|---|---|---|
| content of Zn metal | 0.8% | |
| BET specific surface | 170 | m²/g |
| CTAB specific surface | 170 | m²/g |
| DOP oil uptake | 262 | ml/100 g |
| pH | 8.4 | |
| Na₂SO₄ | 0.7% | |
| content of TiO₂ | 1.7% | |

The particle size characteristics obtained by dry-screening are as follows:

| Mesh size in μm | 250 | 210 | 180 | 150 | 125 |
|---|---|---|---|---|---|
| Cumulative oversize in | 4 | 19 | 43 | 57 | 79 |
| % weight/weight | | | | | |

The particle size characteristics (Laser determination) of the particles obtained are as follows:

| | | |
|---|---|---|
| d₁₀ = | 106 | μm |
| d₅₀ = | 180 | μm |
| d₉₀ = | 273 | μm. |

The results of the cohesion test are as follows:

| Mechanical | d₁₀ | d₅₀ | d₉₀ |
|---|---|---|---|
| stirring time | (μm) | (μm) | (μm) |
| 30 seconds | 106 | 180 | 273 |
| 5 minutes | 45 | 141 | 236 |
| 10 minutes | 27 | 116 | 208 |
| 15 minutes | 20 | 101 | 189 |
| 20 minutes | 17 | 93 | 176 |

CF = 64.4%
compatibility with NaF: 95%

### EXAMPLE 4

The second part of the cake prepared in Example 3 is brought to a loss on ignition of 81.1% by adding water and is kept stirring for 5 hours, after which it is dried using a nozzle sprayer.

The characteristics of the silica microbeads obtained are as follows:

| | | |
|---|---|---|
| content of Zn metal | 0.8% | |
| BET specific surface | 171 | m²/g |
| CTAB specific surface | 170 | m²/g |
| DOP oil uptake | 266 | ml/100 g |
| pH | 8.5 | |
| Na₂SO₄ | 0.7% | |
| content of TiO₂ | 1.7% | |

The particle size characteristics obtained by dry-screening are as follows:

| Mesh size in μm | 250 | 210 | 180 | 150 | 125 |
|---|---|---|---|---|---|
| Cumulative oversize in | 2 | 9 | 29 | 54 | 74 |
| % weight/weight | | | | | |

The particle size characteristics (Laser determination) of the particles obtained are as follows:

| | |
|---|---|
| d₁₀ = | 81 μm |
| d₅₀ = | 157 μm |
| d₉₀ = | 248 μm. |

After dry-screening, the results of the cohesion test are as follows:

| Mechanical | d₁₀ | d₅₀ | d₉₀ |
|---|---|---|---|
| stirring time | (μm) | (μm) | (μm) |
| 30 seconds | 95 | 174 | 269 |
| 5 minutes | 37 | 128 | 224 |
| 10 minutes | 24 | 108 | 202 |
| 15 minutes | 18 | 94 | 179 |
| 20 minutes | 16 | 87 | 171 |

CF = 62.1%
compatibility with NaF: 95%

### EXAMPLE 5

The operation described in Example 3 is repeated, carrying out the treatment step using an aqueous solution containing 85 g/l of ZnSO₄.7H₂O, at a flow rate of 560 l/h for 12 minutes instead of 136 g/l of ZnSO₄.7H₂O at a flow rate of 140 l/h for 12 minutes. The characteristics of the silica microbeads obtained are as follows:

| | |
|---|---|
| content of Zn metal | 2% |
| BET specific surface | 170 m²/g |
| CTAB specific surface | 150 m²/g |
| DOP oil uptake | 258 ml/100 g |
| pH | 8.6 |
| Na₂SO₄ | 0.8% |

The particle size characteristics obtained by dry-screening are as follows:

| Mesh size in μm | 250 | 210 | 180 | 150 | 125 |
|---|---|---|---|---|---|
| Cumulative oversize in | 3 | 19 | 40 | 61 | 79 |
| % weight/weight | | | | | |

The particle size characteristics (Laser determination) of the particles obtained are as follows:

| | |
|---|---|
| d₁₀ = | 85 μm |
| d₅₀ = | 173 μm |
| d₉₀ = | 275 μm. |

The results of the cohesion test are as follows:

| Mechanical | d₁₀ | d₅₀ | d₉₀ |
|---|---|---|---|
| stirring time | (μm) | (μm) | (μm) |
| 30 seconds | 85 | 173 | 275 |
| 5 minutes | 77 | 164 | 259 |
| 10 minutes | 70 | 158 | 250 |
| 15 minutes | 64 | 153 | 245 |
| 20 minutes | 61 | 150 | 243 |

CF = 91.3%
compatibility with NaF: 98%

### COMPARATIVE EXAMPLE 6

The following are introduced into a stainless steel reactor fitted with an impeller stirring system and a heating jacket:

660 liters of water

11.8 kg of Na₂SO₄ (electrolyte)

323 liters of aqueous sodium silicate, with an SiO₂/Na₂O weight ratio equal to 3.45 and a density at 20° C. equal to 1.230.

The SiO₂ concentration in the tail stock is 77 g/l. The mixture is brought to a temperature of 82° C. while stirring is continued. 395 liters of dilute sulfuric acid with a density at 20° C. equal to 1.050 are introduced therein until a pH value (measured at its temperature) equal to 7.5 is obtained in the reaction medium. The reaction temperature is 82° C. during the first 15 minutes of the reaction; it is then raised from 82° C. to 95° C. over about 15 minutes, and then maintained at 95° C. until the end of the reaction.

77 liters of aqueous sodium silicate of the type described above and 106 liters of sulfuric acid, also of the type described above, are then introduced simultaneously into the reaction medium, this simultaneous introduction of acid and silicate being performed such that the pH of the reaction medium during the introduction period is constantly equal to 7.5±0.1.

After introducing all of the silicate, introduction of the dilute acid is continued at a flow rate of 310 l/h, for 5 minutes.

This additional introduction of acid then brings the pH of the medium to a value equal to 5.0.

The total reaction time is set at 85 minutes.

A fumed silica slurry is thus obtained, which is filtered and washed using a filter press, such that a silica cake with a loss on ignition of 79% (i.e. a solids content of 21% by weight) is finally recovered.

This cake is then fluidized by mechanical and chemical action (addition of sodium aluminate). After this crumbling operation, a pumpable cake of pH equal to 6.3 is obtained, which is then atomized by means of a nozzle sprayer.

The characteristics of the silica obtained are as follows:

| BET specific surface | 170 m²/g |
|---|---|
| CTAB specific surface | 160 m²/g |
| DOP oil uptake | 300 ml/100 g |
| pH | 6.7 |
| Na₂SO₄ | 1.2% |

The particle size characteristics obtained by dry-screening are as follows:

| Mesh size in μm | 250 | 210 | 180 | 150 | 125 |
|---|---|---|---|---|---|
| Cumulative oversize in | 15 | 37 | 59 | 77 | 89 |
| % weight/weight | | | | | |

The particle size characteristics (Laser determination) of the particles obtained are as follows:

| | |
|---|---|
| d₁₀ = | 63 μm |
| d₅₀ = | 174 μm |
| d₉₀ = | 285 μm. |

compatibility with NaF: 39%

1. Fumed silica microbeads with sensory properties in the mouth, characterized in that they have:
a CTAB specific surface of at least 100 m²/g, preferably from about 120 m²/g to 250 m²/g and most particularly from about 140 m²/g to 200 m²/g
a DOP oil uptake of at least 200 ml/g, preferably from about 200 ml/g to 350 ml/g
a median particle diameter from 50 μm to 600 μm, generally from about 100 μm to 400 μm
at the surface from 0.5 to 2 parts by weight, preferably from 0.5 to less than 2 parts by weight, most particularly from 0.6 to 1.5 parts by weight (expressed as zinc metal) of a zinc derivative of oxidation state “2” per 100 parts by weight of silica (SiO₂)
a pH from about 7 to 9, preferably from about 7.5 to 8.7.

2. Silica microbeads according to claim 1, characterized in that they have a BET specific surface of at least 100 m²/g, preferably from about 120 m²/g to 300 m²/g, most particularly from about 140 m²/g to 250 m²/g.

3. Silica microbeads according to claim 1 or 2, characterized in that they are compatible with fluorinated compounds, their compatibility being greater than 75%, preferably greater than 85%, with NaF.

4. Silica microbeads according to any one of claims 1 to 3, characterized in that they comprise at least one mineral pigment, in particular a white mineral pigment, in an amount of from about 0.2 to 5 parts by weight, preferably from 0.5 to 4 parts by weight (expressed as weight of pigment), per 100 parts by weight of silica (SiO₂).

5. Process for preparing fumed silica microbeads with sensory properties in the mouth, comprising the steps for formation of an aqueous silica slurry by reacting a silicate of an alkali metal M, with an SiO₂/M₂O ratio from about 2 to 4, with an acidifying agent, separation of the silica slurry formed, washing, fluidization (crumbling) of the silica cake recovered and drying, said process being characterized in that:
the crumbling operation is carried out on a silica cake with a solids content of at least about 15% by weight, said cake resulting from the separation of a silica slurry treated with from 0.5 to 2 parts by weight, preferably from 0.5 to less than 2 parts by weight, most particularly from 0.6 to 1 part by weight (expressed as zinc metal) of at least one acidic or basic soluble zinc compound with an oxidation state of 2 per 100 parts by weight of silica (SiO₂), and performed using a basic agent (in the case of a treatment using at least acidic zinc compound) or acidic agent (in the case of a treatment using at least one basic zinc compound), respectively, at a pH value from about 7.5 to 9.5, preferably from about 7.5 to 9
and the drying operation is carried out by atomization.

6. Process according to claim 5, characterized in that the step for formation of the silica slurry is carried out at a temperature of at least 60° C., preferably from about 70° C. to 98° C., most particularly from 80° C. to 98° C.

7. Process according to claim 5 or 6, characterized in that the step for formation of the slurry is carried out by gradual neutralization of a tail stock consisting of an aqueous alkali metal silicate solution optionally containing an electrolyte, by continuous or batchwise addition of an acid.

8. Process according to claim 5 or 6, characterized in that the step for formation of the slurry is carried out by simultaneously introducing an alkali metal silicate solution and an acid onto a tail stock consisting of:
water optionally with an acid or base added, with a pH from about 4 to 11
or an aqueous alkali metal silicate solution optionally containing an electrolyte, optionally partially neutralized with an acid, or a silica suspension (slurry), with a pH from about 6 to 9 while maintaining a substantially constant pH from about 7 to 9 in the medium during the simultaneous introduction of the reagents,
and then by introducing, optionally, an acid until a pH from about 3 to 6 is obtained.

9. Process according to any one of claims 5 to 8, characterized in that the operation for treatment of the silica slurry is carried out using an acidic zinc compound chosen from soluble inorganic or organic zinc salts of oxidation state “2”.

10. Process according to any one of claims 5 to 9, characterized in that the operation for treatment of the silica slurry carried out using an acidic zinc compound is followed or accompanied by the addition of a basic agent chosen from aqueous ammonia, sodium hydroxide and alkaline silicates.

11. Process according to any one of claims 5 to 8, characterized in that the operation for treatment of the silica slurry is carried out using a basic zinc compound chosen from zincates containing ZnO₂²⁻, HZnO₂⁻, Zn₂O₄⁴⁻ and ZnO₄⁶⁻ anions.

12. Process according to any one of claims 5 to 8 and 11, characterized in that the operation for treatment of the silica slurry carried out using a basic zinc compound is followed by addition of an acidic agent chosen from nitric acid, sulfuric acid, hydrochloric acid and carbonic acid.

13. Process according to any one of claims 5 to 12, characterized in that the treatment operation using at least one soluble zinc compound and the basic or acidic agent for precipitating the zinc derivative is carried out either on the slurry during precipitation or at the end of precipitation and/or after precipitation, before the filtration/washing step.

14. Process according to any one of claims 5 to 10 and 13, characterized in that the treatment operation with the soluble zinc compound is carried out by adding to a silica slurry with a pH from about 7 to 9, preferably from about 7.5 to 8.5, at the end and/or after precipitation at a temperature from about 15° C. to 95° C., an aqueous solution of a soluble acidic zinc salt of oxidation state “2”, followed by a basic agent, until a pH from about 7.5 to 9.5, preferably from about 7.5 to 9, is obtained.

15. Process according to any one of claims 5 to 10 and 13, characterized in that the treatment operation with the soluble zinc compound is carried out by simultaneously adding to a silica slurry during precipitation, with a pH from about 7 to 9, preferably from about 7.5 to 8.5, a silicate solution and an acidic zinc salt solution.

16. Process according to any one of claims 5 to 15, characterized in that the step for separation of the treated slurry is carried out by filtration and is preferably coupled with a washing operation.

17. Process according to claim 16, characterized in that the steps of filtration/washing of the treated slurry are preferably carried out using a filter press and water as washing agent, in order to obtain a silica cake of the desired solids content, of at least 15% by weight, preferably of at least 16% by weight.

18. Process according to any one of claims 5 to 17, characterized in that the step of crumbling of the cake is carried out by the simple action of mechanical stirring using a shearing rotor, optionally with addition of water.

19. Process according to any one of claims 5 to 18, characterized in that the atomization step is carried out using a nozzle sprayer.

20. Process according to any one of claims 5, 6, 8 to 10, 14 and 16 to 19, characterized in that the following are carried out
an operation for formation of a silica slurry by reaction of an alkali metal silicate and an acidifying agent, according to the following steps:
a first step consisting in using an initial tail stock consisting of water, alkali metal silicate and an electrolytic salt, the concentration of alkali metal silicate (expressed as SiO₂) in said tail stock possibly being up to 100 g/l
a second step consisting in neutralizing said tail stock with the acidifying agent until a pH of greater than or equal to about 7, preferably from about 7 to 9.5 and most particularly from about 7 to 8.5, is obtained in the reaction medium;
a third step consisting in introducing into said neutralized tail stock the alkali metal silicate in aqueous solution and the acidifying agent, under conditions such that the pH of the reaction medium remains substantially constant and above or equal to about 7, preferably from about 7 to 9.5 and most particularly from about 7 to 8.5;
an operation for treatment of the slurry obtained, after optional maturation, by addition of 0.5 to 2 parts, preferably from 0.5 to less than 2 parts by weight and most particularly from 0.6 to 1.5 parts (expressed as zinc metal), of at least one soluble acidic zinc compound of oxidation state “2” per 100 parts of silica, and then, after optional maturation, addition of a basic agent, until a pH from about 7.5 to 9.5, preferably from about 7.5 to 9, is obtained in the reaction medium, and optional maturation;
optionally, an operation to adjust the pH to a value from about 7 to 8.5 by addition of an acidic agent, and optional maturation;
an operation of separation and washing so as to obtain a silica cake with a solids content of at least 15% by weight, preferably of at least 16% by weight, in particular using a filter press;
an operation for crumbling by means of mechanical stirrers;
and a spray-drying operation.

21. Process according to any one of claims 5 to 20, characterized in that from about 0.2 to 5 parts by weight, preferably from about 0.5 to 4 parts by weight, per 100 parts by weight of silica, expressed as SiO₂, of at least one mineral pigment, in particular a white mineral pigment, are introduced at any point in the synthesis of the silica slurry or, preferably, during the step of crumbling of the silica cake containing zinc.

22. Use of the silica microbeads forming the subject of any one of claims 1 to 4 or which can be obtained according to the process forming the subject of any one of claims 5 to 21, as sensory agents in the mouth, in toothpaste compositions.

23. Toothpaste compositions comprising the silica microbeads forming the subject of any one of claims 1 to 4 or which can be obtained according to the process forming the subject of any one of claims 5 to 21.

24. Use according to claim 22 or toothpaste compositions according to claim 23, characterized in that the amount of silica microbeads represents from about 0.5% to 20%, preferably from about 1% to 15%, of said compositions.
